(19) United States
(12) Patent Application Publication
(10) Pub. No.: US 2005/0123616 A1
(43) Pub. Date: Jun. 9, 2005

(54) Azithromycin multiparticulate dosage forms by liquid-based processes

(75) Inventors: Leah E. Appel, Bend, OR (US); James B. West, Bend, OR (US); Dwayne T. Friesen, Bend, OR (US); Roderick J. Ray, Bend, OR (US); Marshall D. Crew, Bend, OR (US); David K. Lyon, Bend, OR (US); Scott B. McCray, Bend, OR (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/004,453

(22) Filed: Dec. 3, 2004

Related U.S. Application Data
(60) Provisional application No. 60/527,405, filed on Dec. 4, 2003.

(51) Int. Cl. A61K 9/52; A61K 9/14; B29B 9/00

(52) U.S. Cl. 424/489; 264/5; 514/28

(57) ABSTRACT

Liquid-based processes are disclosed for forming azithromycin multiparticulates having minimal amounts of azithromycin esters.

### BACKGROUND OF THE INVENTION

Multiparticulates are well known dosage forms that comprise a multiplicity of particles whose totality represents the intended therapeutically useful dose of a drug. When taken orally, multiparticulates generally disperse freely in the gastrointestinal tract, maximize absorption, and minimize side effects. See, for example, Multiparticulate Oral Drug Delivery (Marcel Dekker, 1994), and Pharmaceutical Pelletization Technology (Marcel Dekker, 1989).

Azithromycin is the generic name for the drug 9a-aza-9a-methyl-9-deoxo-9a-homoerythromycin A, a broad-spectrum antimicrobial compound derived from erythromycin A. Accordingly, azithromycin and certain derivatives thereof are useful as antibiotics.

It is well known that oral dosing of azithromycin can result in the occurrence of adverse side effects such as cramping, diarrhea, nausea and vomiting. Such side effects are higher at higher doses than at lower doses. Multiparticulates are a known improved dosage form of azithromycin that permit higher oral dosing with relatively reduced side effects. See commonly owned U.S. Pat. No. 6,068,859. Such multiparticulates of azithromycin are particularly suitable for administration of single doses of the drug inasmuch as a relatively large amount of the drug can be delivered at a controlled rate over a relatively long period of time.

The inventors have discovered that certain processes used to form multiparticulates containing azithromycin and use of certain excipients in such multiparticulates can lead to degradation of the azithromycin during and after the process of forming the multiparticulates. The degradation occurs by virtue of a chemical reaction of the azithromycin with the components of the carriers or excipients used in forming the multiparticulates, resulting in the formation of azithromycin esters.

U.S. Pat. No. 6,068,859 discloses several liquid-based processes for forming azithromycin multiparticulates, including extrusion/spheronization, wet granulation, spray-drying, and spray-coating. However, there is no teaching or suggestion as to how to avoid the formation of azithromycin esters likely to form during these processes, nor are any guidelines provided for selecting appropriate excipients and processing conditions for forming multiparticulates having minimal concentrations of azithromycin esters.

What is therefore needed are liquid-based processes wherein the excipients and process conditions are chosen so as to dramatically reduce the formation of azithromycin esters, resulting in a much greater degree of purity of the drug in multiparticulate dosage forms.

### BRIEF SUMMARY OF THE INVENTION

The present invention meets such needs by providing certain liquid-based processes for forming multiparticulates comprising azithromycin and a pharmaceutically acceptable carrier. The processes result in the formation of multiparticulates with minimal concentrations of azithromycin esters, and that are suitable for effecting controlled release of azithromycin. The multiparticulates may be used in azithromycin dosage forms and for treating one in need of azithromycin therapy.

In one aspect, the invention provides a liquid-based process for the formation of multiparticulates comprising the steps: (a) forming a mixture comprising azithromycin, a pharmaceutically acceptable carrier, and a liquid having a boiling point of less than about 150° C.; (b) forming particles from the mixture of step (a) by a method selected from (i) atomization of the mixture, and (ii) coating seed cores with the mixture; and (c) removing a substantial portion of the a liquid from the particles of step (b) to form multiparticulates wherein the following expression is satisfied:
[A]≦0.4/(1−x)
where [A] is the concentration of acid/ester substitution on the carrier in meq/g azithromycin, and x is the weight fraction of the azithromycin in the composition that is crystalline.

The invention also provides methods of treating a patient in need of treatment with azithromycin by administering to the patient a therapeutically effective amount of a pharmaceutical composition comprising azithromycin-containing multiparticulates made by the process of the present invention. The amount of azithromycin which is administered will necessarily be varied according to principles well known in the art, taking into account factors such as the severity of the disease or condition being treated and the size and age of the patient. In general, the drug is to be administered so that an effective dose is received, with the effective dose being determined from safe and efficacious ranges of administration already known for azithromycin.

The invention is particularly useful for administering relatively large amounts of azithromycin to a patient in a single-dose therapy. The amount of azithromycin contained within the multiparticulate dosage form is preferably at least 250 mgA, and can be as high as 7 gA (“mgA” and “gA” mean milligrams and grams of active azithromycin in the dosage form, respectively). The amount contained in the dosage form is preferably about 1.5 to about 4 gA, more preferably about 1.5 to about 3 gA, and most preferably 1.8 to 2.2 gA. For small patients, e.g., children weighing about 30 kg or less, the multiparticulate dosage form can be scaled according to the weight of the patient; in one aspect, the dosage form contains about 30 to about 90 mgA/kg of patient body weight, preferably about 45 to about 75 mgA/kg, more preferably, about 60 mgA/kg.

The multiparticulates formed by the process of the present invention are designed for immediate, sustained, or controlled release of azithromycin after introduction to a use environment. As used herein, a “use environment” can be either the in vivo environment of the GI tract of a mammal, particularly a human, or the in vitro environment of a test solution. Exemplary test solutions include aqueous solutions at 37° C. comprising (1) 0.1 N HCl, simulating gastric fluid without enzymes; (2) 0.01 N HCl, simulating gastric fluid that avoids excessive acid degradation of azithromycin, and (3) 50 mM KH₂PO₄, adjusted to pH 6.8 using KOH, or 50 mM Na₃PO₄, adjusted to pH 6.8 using NaOH, both of which simulate intestinal fluid without enzymes. The inventors have also found that for some formulations, an in vitro test solution comprising 100 mM Na₂HPO₄, adjusted to pH 6.0 using NaOH provides a discriminating means to differentiate among different formulations on the basis of dissolution profile. It has been determined that in vitro dissolution tests in such solutions provide a good indicator of in vivo performance and bioavailability. Further details of in vitro tests and test solutions are described herein.

Detailed guidelines on selection of processing conditions, carriers and their interrelationships are set forth in the Detailed Description of Preferred Embodiments below. Also according to the present invention, reaction rates for excipients may be calculated so as to enable the practitioner to make an informed selection, following the general guideline that an excipient exhibiting a slower rate of ester formation is desirable, while an excipient exhibiting a faster rate of ester formation is undesirable.

### DETAILED DESCRIPTION OF PREFERRED EMBODIMENTS

According to the present invention it has been found that azithromycin ester formation may be dramatically suppressed in a number of ways: (1) by using azithromycin having a high degree of crystallinity; (2) by selection of a carrier from a particular class of materials which exhibit very low rates of ester formation with the drug; (3) by selection of certain processing parameters when a carrier is selected that has inherently higher rates of ester formation; and (4) by using liquids having low degrees of acid/ester substitution.

An acceptable level of azithromycin ester formation is one which, during the time period beginning with formation of multiparticulates and continuing up until dosage, results in the formation of less than about 1 wt % azithromycin esters, meaning the weight of azithromycin esters relative to the total weight of azithromycin originally present in the multiparticulates, preferably less than about 0.5 wt %, more preferably less than about 0.2 wt %, and most preferably less than about 0.1 wt %.

Generally speaking, the class of excipients having inherently low rates of ester formation with azithromycin may be described as pharmaceutically acceptable excipients that contain no or relatively few acid and/or ester substituents as chemical substituents. All references to “acid and/or ester substituents” herein are intended to mean (i) carboxylic acid, sulfonic acid, and phosphoric acid substituents; or (ii) carboxylic acid ester, sulfonyl ester, or phosphate ester substituents, respectively.

Conversely, the class of excipients having inherently higher rates of ester formation with azithromycin may be generally described as pharmaceutically acceptable excipients that contain a relatively greater number of acid and/or ester substituents; within limits, processing conditions for this class of excipients may be utilized to suppress the rate of ester formation to an acceptable level.

In one aspect, at least about 95% of the azithromycin in the multiparticulate is crystalline and the concentration of acid and ester substituents on the carrier is less than about 3.5 meq/g azithromycin. In a second aspect, at least about 90% of the azithromycin in the multiparticulate is crystalline and the concentration of acid and ester substituents on the carrier is less than about 2 meq/g azithromycin. In a third aspect, at least about 80% of the azithromycin in the multiparticulate is crystalline and the concentration of acid and ester substituents on the carrier is less than about 1 meq/g azithromycin.

Azithromycin esters may be formed during the multiparticulate-forming process, during other processing steps required for manufacture of the finished dosage form, or during storage following manufacture but prior to dosing. Since the azithromycin dosage forms may be stored for up to two years or even longer prior to dosing, it is preferred that the concentration of azithromycin esters in the stored dosage form not exceed the above-noted values prior to dosing.

The compositions formed by the process of the present invention comprise “multiparticulates.” The term “multiparticulates” is intended to embrace a dosage form comprising a multiplicity of particles whose totality represents the intended therapeutically useful dose of azithromycin. The particles generally are of a mean diameter from about 40 to about 3000 μm, preferably from about 50 to about 1000 μm, and most preferably from about 100 to about 300 μm. Multiparticulates are preferred because they are amenable to use in scaling dosage forms according to the weight of an individual patient in need of treatment by simply scaling the mass of particles in the dosage form to comport with the patient's weight. They are further advantageous since they allow the incorporation of a large quantity of drug into a simple dosage form such as a sachet that can be formulated into a slurry that can easily be consumed orally. Multiparticulates also have numerous therapeutic advantages over other dosage forms, especially when taken orally, including (1) improved dispersal in the gastrointestinal (GI) tract, (2) more uniform GI tract transit time, and (3) reduced inter- and intrapatient variability.

While the multiparticulates can have any shape and texture, it is preferred that they be spherical, with a smooth surface texture. These physical characteristics lead to excellent flow properties, improved “mouth feel,” ease of swallowing and ease of uniform coating, if required.

Preferably, the azithromycin makes up about 5 wt % to about 90 wt % of the total weight of the multiparticulate, more preferably about 10 wt % to about 80 wt %, even more preferably about 30 wt % to about 60 wt % of the total weight of the multiparticulates.

As used in the present invention, the term “about” means the specified value ±10% of the specified value.

### Liquid-Based Processes

In its broadest sense the liquid-based process useful in forming the azithromycin multiparticulates of the present invention comprises the steps of (a) forming a mixture comprising azithromycin, a pharmaceutically acceptable carrier, and a liquid; (b) forming particles from the mixture of step (a); and (c) removing a substantial portion of the liquid from the particles of step (b) to form the multiparticulates. Preferably, step (b) is conducted by a method selected from (i) atomization of the mixture and (ii) coating seed cores with the mixture.

In the processes of the present invention, a mixture is formed comprising azithromycin, a carrier, and the liquid. The liquid mixture may comprise a solution of azithromycin and carrier both dissolved in the liquid, a suspension of azithromycin in a solution of carrier dissolved in the liquid, a suspension of carrier in a solution of azithromycin dissolved in the liquid, a suspension of both azithromycin and carrier in the liquid, or combinations of these states or any states intermediate such states.

When the crystalline form is a hydrate crystal form it is preferred to add sufficient water to the process liquid to prevent the loss of water from the crystalline drug and thereby keep the azithromycin in its original crystalline form. When the crystalline form is the dihydrate, which is especially preferred, the water concentration should be 30 to 100% of the water solubility in the chosen liquid.

Preferably, the liquid is chosen such that the amount of azithromycin that remains in the crystalline state is maximized. Generally, azithromycin is less reactive when in crystalline form than when dissolved or in amorphous form. In crystalline azithromycin, the azithromycin molecules are locked into a rigid three-dimensional structure that is at a low thermodynamic energy state. Removal of an azithromycin molecule from this crystal structure, for example, to react with a carrier, will therefore take a considerable amount of energy. In addition, crystal forces reduce the mobility of the azithromycin molecules in the crystal structure. The result is that the rate of reaction of azithromycin with acid and ester substituents on a carrier is significantly reduced in crystalline azithromycin when compared to mixtures containing amorphous or dissolved azithromycin.

The liquid used in liquid-based processes to form azithromycin multiparticulates should be sufficiently non-reactive with azithromycin that less than about 1 wt % azithromycin esters are formed, and be pharmaceutically acceptable. As detailed further below, a convenient way to assess the potential for azithromycin to react with a material to form azithromycin esters is to ascertain the material's concentration of acid and ester substituents. Thus, to prevent formation of azithromycin esters by reaction with the liquid, it is preferred that the liquid's concentration of acid and ester substituents be less than about 0.1 meq/g liquid. The term “liquid” is used in its conventional sense, meaning that the material is a fluid having a viscosity of less than about 300 cp at room temperature. Generally, volatile liquids are preferred, since these are easier to remove from the multiparticulates. By “volatile” liquid is meant that the material has a boiling point of less than about 150° C. at ambient pressure, although small amounts of liquids with higher boiling points can be included in mixtures of liquids and still achieve acceptable results.

Examples of liquids suitable for formation of multiparticulates using liquid-based processes include water; alcohols, such as methanol, ethanol, various isomers of propanol and various isomers of butanol; ketones, such as acetone, methyl ethyl ketone and methyl isobutyl ketone; hydrocarbons, such as pentane, hexane, heptane, cyclohexane, methylcyclohexane, octane and mineral oil; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; N,N-dimethylacetamide; acetonitrile; and mixtures thereof.

In one embodiment of the present invention, the liquid selected is one in which azithromycin has a relatively low solubility. The solubility of azithromycin in the liquid is preferably measured at ambient temperature. Low solubility of azithromycin in the liquid tends to limit the amount of amorphous azithromycin present in the composition. Amorphous azithromycin is more reactive than crystalline azithromycin and minimizing amorphous azithromycin in turn minimizes the formation of azithromycin esters. Preferably, the solubility of crystalline azithromycin (such as the dihydrate) in the liquid is less than about 10 mg/mL. Such a low azithromycin solubility in the liquid will ensure that the amount of amorphous azithromycin in the composition is less than about 20 wt %, depending on the liquid-based process used to form the multiparticulate. More preferably, the solubility of azithromycin in the liquid is less than about 5 mg/mL, and most preferably less than about 1 mg/mL. Because azithromycin is a very hydrophilic compound, it has a low solubility in liquids that tend to be relatively hydrophobic. Examples of suitable liquids in which azithromycin has relatively low solubility include hydrocarbons, such as pentane, hexane, heptane, cyclohexane, methylcyclohexane, octane, mineral oil and the like; and hydrophobic ethers, such as methyl tert-butyl ether. When crystalline azithromycin is combined with such liquids it will form a suspension of azithromycin in the liquid.

Although azithromycin is very hydrophilic, the solubility of azithromycin in water is highly pH-dependent, with solubility decreasing with increasing pH. The solubility of crystalline azithromycin dihydrate in distilled water at pH 6.9 is reported to be 1.1 mg/mL. Thus, a preferred liquid for liquid-based processes is water at a pH of 7 or higher. Water with a higher pH can be generated by dissolving a small amount of a base in the water, or by preparing a buffer solution that will precisely control pH.

Examples of bases that can be added to the water to raise the pH include hydroxides, such as sodium hydroxide, calcium hydroxide, ammonium hydroxide, choline hydroxide and potassium hydroxide; bicarbonates, such as sodium bicarbonate, potassium bicarbonate and ammonium bicarbonate; carbonates, such as ammonium carbonate and sodium carbonate; phosphates, such as sodium phosphate and potassium phosphate; borates, such as sodium borate; amines, such as tris(hydroxymethyl)amino methane, ethanolamine, diethanolamine, N-methyl glucamine, glucosaime, ethylenediamine, cyclohexylamine, cyclopentylamine, diethylamine, isopropylamine and triethylamine; proteins, such as gelatin; and amino acids such as lysine, arginine, guanine, glycine and adenine.

A particularly useful buffer is phosphate buffered saline (PBS) solution, which is an aqueous solution comprising 20 mM Na₂HPO₄, 466 mM KH₂PO₄, 87 mM NaCl and 0.2 mM KCl, adjusted to pH 7. Mixtures of such basic and buffered water and a solvent such as an alcohol may be used as well.

Once the mixture comprising azithromycin, a carrier and a liquid is formed, it is formed into particles. Preferably, the particles are formed by a method selected from (i) atomization of the mixture and (ii) coating seed cores with the mixture.

In one embodiment, the particles are formed by atomization of the mixture using an appropriate nozzle to form small droplets of the mixture, which are sprayed into a drying chamber where there is a strong driving force for evaporation of the liquid, to produce solid, generally spherical particles. The strong driving force for evaporation of the liquid is generally provided by maintaining the partial pressure of liquid in the drying chamber well below the vapor pressure of the liquid at the temperature of the particles. This is accomplished by (1) maintaining the pressure in the drying chamber at a partial vacuum (e.g., 0.01 to 0.5 atm); or (2) mixing the droplets with a warm drying gas; or (3) both (1) and (2). Spray-drying processes and spray-drying equipment are described generally in Perry's Chemical Engineers' Handbook, pages 20-54 to 20-57 (6th Ed. 1984).

For example, a suspension is formed comprising 3 to 15 wt % crystalline azithromycin, 3 to 15 wt % carrier such as hydroxypropyl cellulose, and the balance water with a pH of greater than 7. This solution can then be atomized using a two-fluid nozzle into a spray-drying chamber. Drying gas with an inlet temperature of 1500 to 250° C. can be used, with the drying gas outlet temperature being 400 to 80° C., resulting in the formation of multiparticulates. The multiparticulates can then be collected and further dried using procedures well known in the art, such as through the use of tray dryers and microwave dryers. During this process, precautions should be taken to prevent loss of any waters of hydration in a crystalline hydrate, such as the crystalline dihydrate, as outlined above.

In another embodiment, the particles are formed by coating the liquid mixture onto seed cores. The seed cores can be made from any suitable material such as starch, microcrystalline cellulose, sugar or wax, by any known method, such as melt- or spray-congealing, extrusion/spheronization, granulation, spray-drying and the like.

The liquid mixture can be sprayed onto such seed cores using coating equipment known in the pharmaceutical arts, such as pan coaters (e.g., Hi-Coater available from Freund Corp. of Tokyo, Japan, Accela-Cota available from Manesty of Liverpool, U.K.), fluidized bed coaters (e.g., Wurster coaters or top-sprayers available from Glalt Air Technologies of Ramsey, N.J. and from Niro Pharma Systems of Bubendorf, Switzerland) and rotary granulators (e.g., CF-Granulator, available from Freund Corp).

For example, microcrystalline cellulose or sugar seed cores can be coated with a suspension comprising 5 to 15 wt % azithromycin, 2 to 5 wt % carrier, such as hydroxypropyl cellulose, and 93 the balance water with a pH of greater than 7 using a fluidized bed coating apparatus. During the coating process, the conditions are chosen such that the liquid mixture forms a thin coating on the seed cores. While forming this coating, a portion of the liquid is removed from the coating, resulting in formation of a solid coating comprising azithromycin and carrier on the seed core. A subsequent drying process may be used to remove residual liquid from the multiparticulates following the coating step. Sufficient coating solution is applied to the seed cores to result in a multiparticulate containing the desired quantity of azithromycin.

Once the particles are formed, a portion of the liquid is removed, typically in a drying step, thus forming the multiparticulates. Preferably, at least 80% of the liquid is removed from the particles, more preferably at least 90%, and most preferably at least 95% of the liquid is removed from the particle during the drying step. Suitable means for drying include tray dryers, microwave dryers, fluid bed dryers, rotary dryers and spray dryers, all well known in the pharmaceutical arts.

The temperature and humidity used during the drying steps should be selected to minimize the formation of azithromycin esters and to prevent loss in the waters of hydration of crystalline azithromycin. Generally, the drying temperature should not exceed about 50° C. in order to minimize the formation of azithromycin esters. At the same time, the relative humidity should be maintained sufficiently high to avoid loss in the waters of hydration.

The humidity level required is that which is equivalent to or greater than the activity of water in the crystalline state. This can be determined experimentally, for example, using a dynamic vapor sorption apparatus. In this test, a sample of the crystalline azithromycin is placed in a chamber and equilibrated at a constant temperature and relative humidity. The weight of the sample is then recorded. The weight of the sample is then monitored as the relative humidity of the atmosphere in the chamber is decreased. When the relative humidity in the chamber decreases to below the level equivalent to the activity of water in the crystalline state, the sample will begin to lose weight as waters of hydration are lost. Thus, to maintain the crystalline state of the azithromycin, the humidity level should be maintained at or above the relative humidity at which the azithromycin begins to lose weight. A similar test can be used to determine the appropriate amount of solvent vapor required to maintain a crystalline solvate form of azithromycin.

If higher drying temperatures must be used, e.g., greater than 50° C., carriers with somewhat lower concentrations of acid/ester substituents are preferred, since higher drying temperatures increase the rate at which azithromycin esters form.

### Azithromycin

The multiparticulates of the present invention comprise azithromycin.

Preferably, the azithromycin makes up from about 5 wt % to about 90 wt % of the total weight of the multiparticulate, more preferably from about 10 wt % to about 80 wt %, and even more preferably from about 30 wt % to about 60 wt % of the total weight of the multiparticulates.

As used herein, “azithromycin” means all amorphous and crystalline forms of azithromycin including all polymorphs, isomorphs, pseudomorphs, clathrates, salts, solvates and hydrates of azithromycin, as well as anhydrous azithromycin. Reference to azithromycin in terms of therapeutic amounts or in release rates in the claims is to active azithromycin, i.e., the non-salt, non-hydrated azalide molecule having a molecular weight of 749 g/mole.

Preferably, the azithromycin of the present invention is azithromycin dihydrate, which is disclosed in U.S. Pat. No. 6,268,489.

In alternate embodiments of the present invention, the azithromycin comprises a non-dihydrate azithromycin, a mixture of non-dihydrate azithromycins, or a mixture of azithromycin dihydrate and non-dihydrate azithromycins. Examples of suitable non-dihydrate azithromycins include, but are not limited to, alternate crystalline forms B, D, E, F, G, H, J, M, N, O, P, Q and R.

Azithromycin also occurs as Family I and Family II isomorphs, which are hydrates and/or solvates of azithromycin. The solvent molecules in the cavities have a tendency to exchange between solvent and water under specific conditions. Therefore, the solvent/water content of the isomorphs may vary to a certain extent.

Azithromycin form B, a hygroscopic hydrate of azithromycin, is disclosed in U.S. Pat. No. 4,474,768.

Azithromycin forms D, E, F, G, H, J, M, N, O, P, Q and R are disclosed in commonly owned U.S. Patent Publication No. 20030162730, published Aug. 28, 2003.

Forms B, F, G, H, J, M, N, O, and P belong to Family I azithromycin and have a monoclinic P2₁ space group with cell dimensions of a=16.3±0.3 Å, b=16.2±0.3 Å, c=18.4Å0.3 Å and beta=109±20.

Form F azithromycin is an azithromycin ethanol solvate of the formula C₃₈H₇₂N₂O₁₂.H₂O.0.5C₂H₅OH in the single crystal structure and is an az monohydrate hemi-ethanol solvate. Form F is further characterized as containing 2-5 wt % water and 1-4 wt % ethanol by weight in powder samples. The single crystal of form F is crystallized in a monoclinic space group, P2₁ with the asymmetric unit containing two azithromycin molecules, two water molecules, and one ethanol molecule, as a monohydrate/hemi-ethanolate. It is isomorphic to all Family I azithromycin crystalline forms. The theoretical water and ethanol contents are 2.3 and 2.9 wt %, respectively.

Form G azithromycin has the formula C₃₈H₇₂N₂O₁₂.1.5H₂O in the single crystal structure and is an azithromycin sesquihydrate. Form G is further characterized as containing 2.5-6 wt % water and <1 wt % organic solvent(s) by weight in powder samples. The single crystal structure of form G consists of two azithromycin molecules and three water molecules per asymmetric unit, corresponding to a sesquihydrate with a theoretical water content of 3.5 wt %. The water content of powder samples of form G ranges from about 2.5 to about 6 wt %. The total residual organic solvent is less than 1 wt % of the corresponding solvent used for crystallization.

Form H azithromycin has the formula C₃₈H₇₂N₂O₁₂.H₂O.0.5C₃H₈O₂ and may be characterized as an azithromycin monohydrate hemi-1,2 propanediol solvate. Form H is a monohydrate/hemi-propylene glycol solvate of azithromycin free base.

Form J azithromycin has the formula C₃₈H₇₂N₂O₁₂.H₂O.0.5C₃H₇OH in the single crystal structure, and is an azithromycin monohydrate hemi-n-propanol solvate. Form J is further characterized as containing 2-5 wt % water and 1-5 wt % n-propanol by weight in powder samples. The calculated solvent content is about 3.8 wt % n-propanol and about 2.3 wt % water.

Form M azithromycin has the formula C₃₈H₇₂N₂O₁₂.H₂O.0.5C₃H₇OH, and is an azithromycin monohydrate hemi-isopropanol solvate. Form M is further characterized as containing 2-5 wt % water and 1-4 wt % 2-propanol by weight in powder samples. The single crystal structure of form M would be a monohydrate/hemi-isopropranolate.

Form N azithromycin is a mixture of isomorphs of Family I. The mixture may contain variable percentages of isomorphs F, G, H, J, M and others, and variable amounts of water and organic solvents, such as ethanol, isopropanol, n-propanol, propylene glycol, acetone, acetonitrile, butanol, pentanol, etc. The weight percent of water can range from 1-5.3 wt % and the total weight percent of organic solvents can be 2-5 wt % with each solvent making up 0.5-4 wt %.

Form O azithromycin has the formula C₃₈H₇₂N₂O₁₂.0.5H₂O.0.5C₄H₉and is a hemihydrate hemi-n-butanol solvate of azithromycin free base by single crystal structural data.

Form P azithromycin has the formula C₃₈H₇₂N₂O₁₂.H₂O.0.5C₅H₁₂O and is an azithromycin monohydrate hemi-n-pentanol solvate.

Form Q is distinct from Families I and II, has the formula C₃₈H₇₂N₂O₁₂.H₂O.0.5C₄H₈O and is an azithromycin monohydrate hemi-tetrahydrofuran (THF) solvate. It contains about 4% water and about 4.5 wt % THF.

Forms D, E and R belong to Family II azithromycin and contain an orthorhombic P2₁ 2₁2₁ space group with cell dimensions of a=8.9±0.4 Å, b=12.3±0.5 Å and c=45.8±0.5 Å.

Form D azithromycin has the formula C₃₈H₇₂N₂O₁₂.H₂O.C₆H₁₂ in its single crystal structure, and is an azithromycin monohydrate monocyclohexane solvate.

Form D is further characterized as containing 2-6 wt % water and 3-12 wt % cyclohexane by weight in powder samples. From single crystal data, the calculated water and cyclohexane content of form D is 2.1 and 9.9 wt %, respectively.

Form E azithromycin has the formula C₃₈H₇₂N₂O₁₂.H₂O.C₄H₈O and is an azithromycin monohydrate mono-THF solvate by single crystal analysis.

Form R azithromycin has the formula C₃₈H₇₂N₂O₁₂.H₂O.C₅H₁₂O and is an azithromycin monohydrate mono-methyl tert-butyl ether solvate. Form R has a theoretical water content of 2.1 wt % and a theoretical methyl tert-butyl ether content of 10.3 wt %.

Other examples of non-dihydrate azithromycin include, but are not limited to, an ethanol solvate of azithromycin or an isopropanol solvate of azithromycin. Examples of such ethanol and isopropanol solvates of azithromycin are disclosed in U.S. Pat. Nos. 6,365,574 and 6,245,903 and U.S. patent application Publication No. 20030162730, published Aug. 28, 2003.

Additional examples of non-dihydrate azithromycin include, but are not limited to, azithromycin monohydrate as disclosed in U.S. patent application Publication Nos. 20010047089, published Nov. 29, 2001, and 20020111318, published Aug. 15, 2002, as well as International Application Publication Nos. WO 01/00640, WO 01/49697, WO 02/10181 and WO 02/42315.

Further examples of non-dihydrate azithromycin include, but are not limited to, anhydrous azithromycin as disclosed in U.S. patent application Publication No. 20030139583, published Jul. 24, 2003, and U.S. Pat. No. 6,528,492.

Examples of suitable azithromycin salts include, but are not limited to, the azithromycin salts as disclosed in U.S. Pat. No. 4,474,768.

Preferably, at least 70% of the azithromycin in the multiparticulates is crystalline. The degree of azithromycin crystallinity in the multiparticulates can be “substantially crystalline,” meaning that the amount of crystalline azithromycin in the multiparticulates is at least about 80%, “almost completely crystalline,” meaning that the amount of crystalline azithromycin is at least about 90%, or “essentially crystalline,” meaning that the amount of crystalline azithromycin in the multiparticulates is at least 95%.

The crystallinity of azithromycin in the multiparticulates is determined using Powder X-Ray Diffraction (PXRD) analysis. In an exemplary procedure, PXRD analysis may be performed on a Bruker AXS D8 Advance diffractometer. In this analysis, samples of about 500 mg are packed in Lucite sample cups and the sample surface smoothed using a glass microscope slide to provide a consistently smooth sample surface that is level with the top of the sample cup. Samples are spun in the φ plane at a rate of 30 rpm to minimize crystal orientation effects. The X-ray source (S/B KCu_{α}, λ=1.54 Å) is operated at a voltage of 45 kV and a current of 40 mA. Data for each sample are collected over a period of 20 to 60 minutes in continuous detector scan mode at a scan speed of 12 seconds/step and a step size of 0.02°/step. Diffractograms are collected over the 2θ range of 10° to 16°.

The crystallinity of the test sample is determined by comparison with calibration standards as follows. The calibration standards consist of physical mixtures of 20 wt %/80 wt % azithromycin/carrier, and 80 wt %/20 wt % azithromycin/carrier. Each physical mixture is blended together 15 minutes on a Turbula mixer. Using the instrument software, the area under the diffractogram curve is integrated over the 2θ range of 10° to 16° using a linear baseline. This integration range includes as many azithromycin-specific peaks as possible while excluding carrier-related peaks. In addition, the large azithromycin-specific peak at approximately 10° 2θ is omitted due to the large scan-to-scan variability in its integrated area. A linear calibration curve of percent crystalline azithromycin versus the area under the diffractogram curve is generated from the calibration standards. The crystallinity of the test sample is then determined using these calibration results and the area under the curve for the test sample. Results are reported as a mean percent azithromycin crystallinity (by crystal mass).

Crystalline azithromycin is preferred since it is more chemically and physically stable than the amorphous form. The chemical stability arises from the fact that in crystalline form, azithromycin molecules are locked into a rigid three-dimensional structure that is at a low thermodynamic energy state. Removal of an azithromycin molecule from this structure, for example, to react with a carrier, will therefore take a considerable amount of energy. In addition, crystal forces reduce the mobility of the azithromycin molecules in the crystal structure. The result is that the rate of reaction of azithromycin with acid and ester substituents on a carrier is. significantly reduced in crystalline azithromycin when compared to formulations containing amorphous azithromycin.

### Formation of Azithromycin Esters

Azithromycin esters can form either through direct esterification or transesterification of the hydroxyl substituents of azithromycin. By direct esterification is meant that an excipient having a carboxylic acid moiety can react with the hydroxyl substituents of azithromycin to form an azithromycin ester. By transesterification is meant that an excipient having an ester substituent can react with the hydroxyl groups, transferring, e.g., the carboxylate of the carrier to azithromycin, also resulting in an azithromycin ester. Purposeful synthesis of azithromycin esters has shown that the esters typically form at the hydroxyl group attached to the 2′ carbon (C2′) of the desosamine ring; however esterification at the hydroxyls attached to the 4″ carbon on the cladinose ring (C4″) or the hydroxyls attached to the C6, C11, or C12 carbons on the macrolide ring may also occur in azithromycin formulations. An example of a transesterification reaction of azithromycin with a C₁₆ to C₂₂ fatty acid glyceryl triester is shown below.

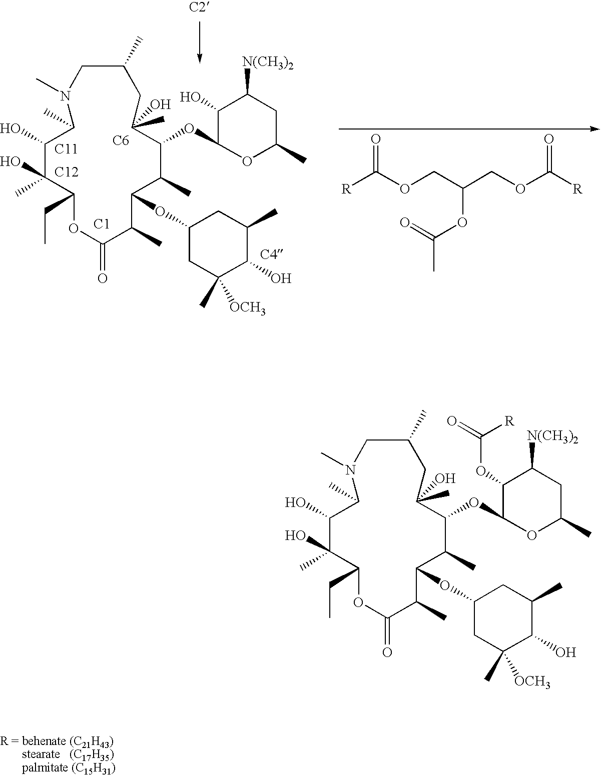

Typically in such reactions, one acid or one ester substituent on the excipient can each react with one molecule of azithromycin, although formation of two or more esters on a single molecule of azithromycin is possible. One convenient way to assess the potential for an excipient to react with azithromycin to form an azithromycin ester is the number of moles or equivalents of acid or ester substituents on the carrier per gram of azithromycin in the composition. For example, if an excipient has 0.13 milliequivalents (meq) of acid or ester substituents per gram of azithromycin in the composition and all of these acid or ester substituents reacted with azithromycin to form mono-substituted azithromycin esters, then 0.13 meq of azithromycin esters would form. Since the molecular weight of azithromycin is 749 g/mole, this means that 0.1 g of azithromycin would be converted to an azithromycin ester in the composition for every gram of azithromycin initially present in the composition. Thus, the concentration of azithromycin esters in the multiparticulates would be 1 wt %. However, it is unlikely that every acid and ester substituent in a composition will react to form azithromycin esters. As noted below, the greater the crystallinity of azithromycin in the multiparticulate, the greater can be the concentration of acid and ester substituents on the excipient and still result in a composition with acceptable amounts of azithromycin esters.

The rate of azithromycin ester formation Rₑ in wt %/day for a given excipient may be predicted using a zero-order reaction model, according to the following equation:
Rₑ=Cₑₛₜₑᵣₛ÷t (I)
where Cₑₛₜₑᵣₛ is the concentration of azithromycin esters formed (wt %), and t is time of contact between azithromycin and the excipient in days at temperature T (° C.).

A variety of azithromycin esters may be formed by reaction of the excipient with the azithromycin. Unless otherwise stated, Cₑₛₜₑᵣₛ refers to the concentration of all azithromycin esters combined.

One procedure for determining the reaction rate for forming azithromycin esters with the excipient is as follows. The excipient is heated to a constant temperature above its melting point and an equal weight of azithromycin is added to the molten excipient, thereby forming a suspension or solution of azithromycin in the molten excipient. Samples of the molten mixture are then periodically withdrawn and analyzed for formation of azithromycin esters using the procedures described below. The rate of ester formation can then be determined using equation (I) above.

Alternatively, the excipient and azithromycin can be blended at a temperature below the melting temperature of the excipient and the blend stored at a convenient temperature, such as 50° C. Samples of the blend can be periodically removed and analyzed for azithromycin esters, as described below. The rate of ester formation can then be determined using equation (I) above.

A number of methods well known in the art can be used to determine the concentration of azithromycin esters in multiparticulates. An exemplary method is by high performance liquid chromatography/mass spectrometer (LC/MS) analysis. In this method, the azithromycin and any azithromycin esters are extracted from the multiparticulates using an appropriate solvent, such as methanol or isopropyl alcohol. The extraction solvent may then be filtered with a 0.45 μm nylon syringe filter to remove any particles present in the solvent. The various species present in the extraction solvent can then be separated by high performance liquid chromatography (HPLC) using procedures well known in the art. A mass spectrometer is used to detect species, with the concentrations of azithromycin and azithromycin esters being calculated from the mass-spectrometer peak areas based on either an internal or external azithromycin control. Preferably, if authentic standards of the azithromycin esters have been synthesized, external references to the azithromycin esters may be used. The azithromycin ester value is then reported as a percentage of the total azithromycin in the sample.

Compositions prepared by the processes of the present invention have less than about 1 wt % total azithromycin esters after storage for 2 years at ambient temperature and humidity or, under ICH guidelines, 25° C. and 600 relative humidity (RH). Preferred embodiments of the invention have less than about 0.5 wt % azithromycin esters after such storage, more preferably less than about 0.2 wt %, and most preferably less than about 0.1 wt %.

Accelerated storage tests can be performed following International Conference on Harmonization (ICH) guidelines. Under these guidelines, a simulation of two years at ambient temperature is conducted by measuring the ester formation of a sample stored for one year at 30° C./60% relative humidity (RH). More rapid simulations can be conducted by storing the sample for six months at 40° C./75% RH.

To satisfy a total azithromycin esters content of less than about 1 wt %, the rate of total azithromycin esters formation should be
Rₑ≦3.6×10⁷·e^{-7070/(T+273)}
where T is the temperature in ° C.

To satisfy the preferred total azithromycin esters content of less than about 0.5 wt %, rate of total azithromycin esters formation should be
Rₑ≦1.8×10⁷·e^{-7070/(T+273)}.

To satisfy the more preferred total azithromycin esters content of less than about 0.2 wt %, the rate of total azithromycin esters formation should be
Rₑ≦7.2×10⁶·e^{-7070/(T+273)}.

To satisfy the most preferred total azithromycin esters content of less than about 0.1 wt %, the rate of total azithromycin esters formation should be
Rₑ≦3.6×10⁶·e^{-70701/(+273)}.

A convenient way to assess the potential for azithromycin to react with an excipient to form azithromycin esters is to ascertain the excipient's degree of acid/ester substitution. This can be determined by dividing the number of acid and ester substituents on each excipient molecule by the molecular weight of each excipient molecule, yielding the number of acid and ester substituents per gram of each excipient molecule. As many suitable excipients are actually mixtures of several specific molecule types, average values of numbers of substituents and molecular weight may be used in these calculations. The concentration of acid and ester substituents per gram of azithromycin in the composition may then be determined by multiplying this number by the mass of excipient in the composition and dividing by the mass of azithromycin in the composition. For example, glyceryl monostearate,
CH₃(CH₂)₁₆COOCH₂CHOHCH₂OH
has a molecular weight of 358.6 g/mol and one ester substituent per mole. Thus, the ester substituents concentration per gram of excipient is 1 eq÷358.6 g, or 0.0028 eq/g excipient or 2.8 meq/g excipient. If a multiparticulate is formed containing 30 wt % azithromycin and 70 wt % glyceryl monostearate, the ester substituent concentration per gram of azithromycin would be
2.8 meq/g×70/30=6.5 meq/g azithromycin.
Calculations like the one above can be used to calculate the concentration of acid and ester substituents on any excipient candidate.

However, in most cases, the excipient candidate is not available in pure form, and may constitute a mixture of several primary molecular types as well as small amounts of impurities or degradation products that could be acids or esters. In addition, many excipient candidates are natural products or are derived from natural products that may contain a wide range of compounds, making the above calculations extremely difficult, if not impossible. For these reasons, the inventors have found that the degree of acid/ester substitution on such materials can often most easily be estimated by using the Saponification Number or Saponification Value of the excipient. The Saponification Number is the number of milligrams of potassium hydroxide required to neutralize or hydrolyze any acid or ester substituents present in 1 gram of the material. Measurement of the Saponification Number is a standard way to characterize many commercially available excipients and the manufacturer often provides their Saponification Number. The Saponification Number will not only account for acid and ester substituents present on the excipient itself, but also for any such substituents present due to impurities or degradation products in the excipient. Thus, the Saponification Number will often provide a more accurate measure of the degree of acid/ester substitution in the excipient.

One procedure for determining the Saponification Number of a candidate excipient is as follows. A potassium hydroxide solution is prepared by first adding 5 to 10 g of potassium hydroxide to one liter of 95% ethanol and boiling the mixture under a reflux condenser for about an hour. The ethanol is then distilled and cooled to below 15.5° C. While keeping the distilled ethanol below this temperature, 40 g of potassium hydroxide is dissolved in the ethanol, forming the alkaline reagent. A 4 to 5 g sample of the excipient is then added to a flask equipped with a refluxing condenser. A 50 mL sample of the alkaline reagent is then added to the flask and the mixture is boiled under refluxing conditions until saponification is complete, generally, about an hour. The solution is then cooled and 1 ml of phenolphthalein solution (1% in 95% ethanol) is added to the mixture and the mixture titrated with 0.5 N HCl until the pink color just disappears. The Saponification Number in milligrams of potassium hydroxide per gram of material is then calculated from the following equation:
Saponification Number=[28.05×(B−S)]÷weight of sample
where B is the number of mL of HCl required to titrate a blank sample (a sample containing no excipient) and S is the number of mL of HCl required to titrate the sample. Further details of such a method for determining the Saponification Number of a material is given in Welcher, Standard Methods of Chemical Analysis (1975). The American Society for Testing and Materials (ASTM) also has established several tests for determining the Saponification Number for various materials, such as ASTM D1387-89, D94-00, and D558-95. These methods may also be appropriate for determining the Saponification Number for a potential excipient.

For some excipients, the processing conditions used to form the multiparticulates (e.g., high temperature) may result in a change in the chemical structure of the excipient, possibly leading to the formation of acid and/or ester substituents, e.g., by oxidation. Thus, the Saponification Number of a excipient should be measured after it has been exposed to the processing conditions anticipated for forming the multiparticulates. In this way, potential degradation products from the excipient that may result in the formation of azithromycin esters can be accounted for.

The degree of acid and ester substitution on an excipient can be calculated from the Saponification Number as follows. Dividing the Saponification number by the molecular weight of potassium hydroxide, 56.11 g/mol, results in the number of millimoles of potassium hydroxide required to neutralize or hydrolyze any acid or ester substituents present in one gram of the excipient. Since one mole of potassium hydroxide will neutralize one equivalent of acid or ester substituents, dividing the Saponification Number by the molecular weight of potassium hydroxide also results in the number of milliequivalents (meq) of acid or ester substituents present in one gram of excipient.

For example, glyceryl monostearate can be obtained with a Saponification Number of 165, as specified by the manufacturer. Thus, the degree of acid/ester substitution per gram of excipient or its acid/ester concentration is
165 meq/g÷56.11 =2.9 meq/g excipient.

Using the above example of a composition with 30 wt % azithromycin and 70 wt % glyceryl monostearate, the theoretical concentration of esters formed per gram of azithromycin if all of the azithromycin reacted would be
2.9 meq/g×70/30=6.8 meq/g.

When the multiparticulate comprises two or more excipients, the total concentration of acid and ester groups in all excipients should be used to determine the degree of acid/ester substitution per gram of azithromycin in the multiparticulates. For example, if excipient A has a concentration of acid/ester substituents [A] of 3.5 meq/g azithromycin present in the composition and excipient B has an [A] of 0.5 meq/g azithromycin, and both are present in an amount of 50 wt % of the total amount of excipient in the composition, then the mixture of excipients has an effective [A] of (3.5+0.5)÷2, or 2.0 meq/g azithromycin. In this manner some excipients having much higher degrees of acid/ester substitution may be used in the composition.

Carriers and excipients useful in the present invention can be classified into four general categories (1) non-reactive; (2) low reactivity; (3) moderate reactivity; and (4) highly reactive in relation to their tendency to form azithromycin esters.

Non-reactive carriers and excipients generally have no acid or ester substituents and are free from impurities that contain acids or esters. Generally, non-reactive materials will have an acid/ester concentration of less than 0.0001 meq/g excipient. Non-reactive carriers and excipients are very rare since most materials contain small amounts of impurities. Non-reactive carriers and excipients must therefore be highly purified. In addition, non-reactive carriers and excipients are often hydrocarbons, since the presence of other elements in the excipient can lead to acid or ester impurities. The rate of formation of azithromycin esters for non-reactive carriers and excipients is essentially zero, with no azithromycin esters forming under the conditions described above for determining the azithromycin reaction rate with an excipient. Examples of non-reactive carriers and excipients include highly purified forms of the following hydrocarbons: synthetic wax, microcrystalline wax, and paraffin wax.

Low reactivity carriers and excipients also do not have acid or ester substituents, but often contain small amounts of impurities or degradation products that contain acid or ester substituents. Generally, low reactivity carriers and excipients have an acid/ester concentration of less than about 0.1 meq/g of excipient. Generally, low reactivity carriers and excipients will have a rate of formation of azithromycin esters of less than about 0.005 wt %/day when measured at 100° C. Examples of low reactivity carriers and excipients include long-chain alcohols, such as stearyl alcohol, cetyl alcohol, and polyethylene glycol; poloxamers (block copolymers of ethylene oxide and propylene oxide); ethers, such as polyoxyethylene alkyl ethers; ether-substituted cellulosics, such as hydroxypropyl cellulose, hydroxypropyl methyl cellulose, and ethylcellulose; sugars, such as glucose, sucrose, xylitol, sorbitol and maltitol; and salts, such as sodium chloride, potassium chloride, lithium chloride, calcium chloride, magnesium chloride, sodium sulfate, potassium sulfate, sodium carbonate, magnesium sulfate and potassium phosphate.

Moderate reactivity carriers and excipients often contain acid or ester substituents, but relatively few as compared to the molecular weight of the excipient. Generally, moderate reactivity carriers and excipients have an acid/ester concentration of about 0.1 to about 3.5 meq/g of excipient. Examples include long-chain fatty acid ester, such as glyceryl monooleate, glyceryl monostearate, glyceryl palmitostearate, polyethoxylated castor oil derivatives, glyceryl dibehenate, and mixtures of mono-, di-, and trialkyl glycerides including mixtures of mono-, di-, and tribehenate, glyceryl tristearate, glyceryl tripalmitate and hydrogenated vegetable oils; glycolized fatty acid esters, such as polyethylene glycol stearate and polyethylene glycol distearate; polysorbates; and waxes, such as carnauba wax and white and yellow beeswax.

Highly reactive carriers and excipients usually have several acid or ester substituents or low molecular weights. Generally, highly reactive carriers and excipients have an acid/ester concentration of more than about 3.5 meq/g of excipient and have a rate of formation of azithromycin esters of more than about 40 wt %/day at 100° C. Examples include carboxylic acids such as stearic acid, alginic acid, benzoic acid, citric acid, fumaric acid, lactic acid, and maleic acid; short to medium chain fatty-acid esters, such as isopropyl palmitate, isopropyl myristate, triethyl citrate, lecithin, triacetin, and dibutyl sebacate; ester-substituted cellulosics, such as cellulose acetate, cellulose acetate phthalate, hydroxypropyl methyl cellulose phthalate, cellulose acetate trimellitate, and hydroxypropyl methyl cellulose acetate succinate (HPMCAS); and acid or ester functionalized polymethacrylates and polyacrylates. Note that the reactivity of the polymeric carriers and excipients listed above will depend on the degree of substitution of any acid and ester substituent on the polymer. For example, Shin Etsu (Japan) makes several grades of HPMCAS. The HPMCAS-HF grade contains about 3.2 meq/g excipient of acetate and succinate substituents, while the HPMCAS-MF grade contains about 8.3 meq/g excipient. Thus, some of these polymers may have a moderate reactivity.

Generally, the acid/ester concentration on highly reactive carriers and excipients (e.g., greater than about 3.5 meq/g) is so high that if these excipients come into direct contact with azithromycin in the formulation, unacceptably high concentrations of azithromycin esters form during processing or storage of the composition. Thus, such highly reactive carriers and excipients are preferably only used in combination with a carrier or excipient with lower reactivity so that the total amount of acid and ester groups on the carrier or excipient used in the multiparticulate is low.

To obtain multiparticulates having acceptable concentrations of azithromycin esters of less than about 1 wt % azithromycin esters, the inventors have found there is a trade-off relationship between the crystallinity of azithromycin in the multiparticulate and the concentration of acid and ester substituents on the carrier and optional excipients. Generally speaking, the higher the crystallinity of the azithromycin in the composition, the higher may be the degree of acid/ester substitution on the carrier and optional excipients to obtain multiparticulates with acceptable amounts of azithromycin esters.

The trade-off relationship between azithromycin crystallinity and the degree of acid/ester substitution of the carrier and optional excipients may be quantified by the following mathematical expression:
[A]≦0.04/(1−x) (II)
where [A] is the concentration of acid/ester substitution on the carrier and optional excipients in meq/g azithromycin, and x is the weight fraction of the azithromycin in the composition that is crystalline. Preferably, the azithromycin and carrier/excipient will satisfy the following expression:
[A]≦0.02/(1−x). (III)

More preferably, azithromycin and carrier/excipient will satisfy the following expression:
[A]≦0.008/(1−x). (IV)

Most preferably, the azithromycin and carrier/excipient will satisfy the following expression:
[A]≦0.004/(1−x). (V)

### Carriers

The multiparticulates comprise a pharmaceutically acceptable carrier. By “pharmaceutically acceptable” is meant the carrier must be compatible with the other ingredients of the composition, and not deleterious to the recipient thereof. The carrier functions as a matrix for the multiparticulate or to affect the rate of release of azithromycin from the multiparticulate, or both. Carriers will generally make up about 10 wt % to about 95 wt % of the multiparticulate, preferably about 20 wt % to about 90 wt % of the multiparticulate, and more preferably about 40 wt % to about 70 wt % of the multiparticulates, based on the total mass of the multiparticulate. The carrier is preferably solid at temperatures of about 40° C. The inventors have found that if the carrier is not a solid at 40° C., there can be changes in the physical characteristics of the composition over time, especially when stored at elevated temperatures, such as at 40° C. Thus, it is preferred that the carrier be a solid at a temperature of about 500, more preferably about 60° C.

Examples of carriers suitable for use in the multiparticulates of the present invention include waxes, such as synthetic wax, microcrystalline wax, paraffin wax, carnauba wax, and beeswax; glycerides, such as glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate; long-chain alcohols, such as stearyl alcohol, cetyl alcohol, and polyethylene glycol; and mixtures thereof.

### Optional Excipients

The multiparticulates may optionally include excipients to aid in forming the multiparticulates, to affect the release rate of azithromycin from the multiparticulates, or for other purposes known in the art.

The multiparticulates may optionally include a dissolution enhancer. Dissolution enhancers increase the rate of dissolution of the drug from the carrier. In general, dissolution enhancers are amphiphilic compounds and are generally more hydrophilic than the carrier. Dissolution enhancers will generally make up about 0.1 to about 30 wt % of the total mass of the multiparticulate. Generally, the rate of release of azithromycin from the composition increases with increasing amounts of dissolution enhancers present. Such agents generally have a high water solubility and are often surfactants or wetting agents that can promote solubilization of other excipients in the composition. Exemplary dissolution enhancers include alcohols such as stearyl alcohol, cetyl alcohol, and polyethylene glycol; surfactants, such as poloxamers (such as poloxamer 188, poloxamer 237, poloxamer 338, and poloxamer 407), docusate salts, polyoxyethylene alkyl ethers, polyoxyethylene castor oil derivatives, polysorbates, polyoxyethylene alkyl esters, sodium lauryl sulfate, and sorbitan monoesters; sugars such as glucose, sucrose, xylitol, sorbitol, and maltitol; salts such as sodium chloride, potassium chloride, lithium chloride, calcium chloride, magnesium chloride, sodium sulfate, potassium sulfate, sodium carbonate, magnesium sulfate, and potassium phosphate; amino acids such as alanine and glycine; and mixtures thereof. Preferably, the dissolution enhancer is at least one surfactant, and most preferably, the dissolution enhancer is at least one poloxamer.

While not wishing to be bound by any particular theory or mechanism, it is believed that dissolution enhancers present in the multiparticulates affect the rate at which the aqueous use environment penetrates the multiparticulate, thus affecting the rate at which azithromycin is released. In addition, such agents may enhance the azithromycin release rate by aiding in the aqueous dissolution of the carrier itself, often by solubilizing the carrier in micelles. Further details of dissolution enhancers and selection of appropriate excipients for azithromycin multiparticulates are disclosed in commonly assigned U.S. patent application Ser. No. ______ (“Controlled Release Multiparticulates Formed with Dissolution Enhancers,” Attorney Docket No. PC25016), filed concurrently herewith.

Agents that inhibit or delay the release of azithromycin from the multiparticulates can also be included in the carrier. Such dissolution-inhibiting agents are generally hydrophobic. Examples of dissolution-inhibiting agents include hydrocarbon waxes, such as microcrystalline and paraffin wax and polyethylene glycols having molecular weights greater than about 20,000 daltons.

Other excipients may be added to adjust the release characteristics of the multiparticulates or to improve processing and will typically make up 0 to 50 wt % of the multiparticulate, based on the total mass of the multiparticulate. For example, since the solubility of azithromycin in aqueous solution decreases with increasing pH, a base may be included in the composition to decrease the rate at which azithromycin is released in an aqueous use environment. Examples of bases that can be included in the composition include di- and tri-basic sodium phosphate, di- and tri-basic calcium phosphate, mono-, di-, and tri-ethanolamine, sodium bicarbonate, sodium citrate dihydrate as well as other oxide, hydroxide, phosphate, carbonate, bicarbonate and citrate salts, including various hydrated and anhydrous forms known in the art.

Still other excipients may be added to reduce the static charge on the multiparticulates; examples of such anti-static agents include talc and silicon dioxide.

Flavorants, colorants, and other excipients may also be added in their usual amounts for their usual purposes.

In one embodiment, the multiparticulate comprises about 20 to about 75 wt % azithromycin, about 25 to about 80 wt % of a carrier, and about 0.1 to about 30 wt % of a dissolution enhancer based on the total mass of the multiparticulate.

In a more preferred embodiment, the multiparticulate comprises about 35 wt % to about 55 wt % azithromycin; about 40 wt % to about 65 wt % of an excipient selected from waxes, such as synthetic wax, microcrystalline wax, paraffin wax, carnauba wax, and beeswax; glycerides, such as glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate and mixtures thereof; and about 0.1 to about 15 wt % of a dissolution enhancer selected from surfactants, such as poloxamers, polyoxyethylene alkyl ethers, polyethylene glycol, polysorbates, polyoxyethylene alkyl esters, sodium lauryl sulfate, and sorbitan monoesters; alcohols, such as stearyl alcohol, cetyl alcohol and polyethylene glycol; sugars, such as glucose, sucrose, xylitol, sorbitol and maltitol; salts, such as sodium chloride, potassium chloride, lithium chloride, calcium chloride, magnesium chloride, sodium sulfate, potassium sulfate, sodium carbonate, magnesium sulfate and potassium phosphate; amino acids, such as alanine and glycine; and

mixtures thereof.

In another embodiment, the multiparticulates made by the process of the present invention comprise (a) azithromycin; (b) a glyceride carrier having at least one alkylate substituent of 16 or more carbon atoms; and (c) a poloxamer. At least 70 wt % of the drug in the multiparticulate is crystalline. The choice of these particular carrier excipients allows for precise control of the release rate of the azithromycin over a wide range of release rates. Small changes in the relative amounts of the glyceride carrier and the poloxamer dissolution enhancer result in large changes in the release rate of the drug. This allows the release rate of the drug from the multiparticulate to be precisely controlled by selecting the proper ratio of drug, glyceride and poloxamer. These matrix materials have the further advantage of releasing nearly all of the drug from the multiparticulate. Such multiparticulates are disclosed more fully in commonly assigned U.S. patent application Ser. No. ______ (“Multiparticulate Crystalline Drug Compositions Having Controlled Release Profiles,” Attorney Docket No. PC25020), filed concurrently herewith.

In one aspect, the multiparticulates are in the form of a non-disintegrating matrix. By “non-disintegrating matrix” is meant that at least a portion of the carrier does not dissolve or disintegrate after introduction of the multiparticulate to an aqueous use environment. In such cases, the azithromycin and optionally a portion of the carriers or optional excipients, for example, a dissolution enhancer, are released from the multiparticulate by dissolution. At least a portion of the carrier does not dissolve or disintegrate and is excreted when the use environment is in vivo, or remains suspended in a test solution when the use environment is in vitro. In this aspect, it is preferred that the carrier have a low solubility in the aqueous use environment. Preferably, the solubility of the carrier in the aqueous use environment is less than about 1 mg/mL, more preferably less than about 0.1 mg/mL, and most preferably less than about 0.01 mg/mL. Examples of suitable low solubility carriers include waxes, such as synthetic wax, microcrystalline wax, paraffin wax, carnauba wax, and beeswax; glycerides, such as glyceryl monooleate, glyceryl monostearate, glyceryl palm itostearate, glyceryl mono-, di- or tribehenates, glyceryl tristearate, glyceryl tripalmitate and mixtures thereof.

### Controlled Release

While the azithromycin multiparticulates made by the inventive process are suitable for immediate, sustained, or controlled release of the drug, they are particularly well-suited for controlled release of azithromycin after introduction to a use environment. The multiparticulates can advantageously effect the release of azithromycin at a rate sufficiently slow to ameliorate side effects. The multiparticulates can also release the bulk of the azithromycin in the portion of the GI tract distal to the duodenum. In the following, reference to “azithromycin” in terms of therapeutic amounts or in release rates is to active azithromycin, i.e., the non-salt, non-hydrated macrolide molecule having a molecular weight of 749 g/mol.

In one aspect, the compositions formed by the inventive process release azithromycin according to the release profiles set forth in commonly assigned U.S. Pat. No. 6,068,859.

In another aspect, the compositions formed by the inventive process, following administration of a dosage form containing the composition to a stirred buffered test medium comprising 900 mL of pH 6.0 Na₂HPO₄ buffer at 37° C., releases azithromycin to the test medium at the following rate: (i) from about 15 to about 55 wt %, but no more than 1.1 gA of the azithromycin in the dosage form at 0.25 hour; (ii) from about 30 to about 75 wt %, but no more than 1.5 gA, preferably no more than 1.3 gA of the azithromycin in the dosage form at 0.5 hour; and (iii) greater than about 50 wt % of the azithromycin in the dosage form at 1 hour after administration to the buffered test medium. In addition, dosage forms containing the inventive compositions exhibit an azithromycin release profile for an patient in a fasted state that achieves a maximum azithromycin blood concentration of at least 0.5 μg/mL in at least 2 hours from dosing and an area under the azithromycin blood concentration versus time curve of at least 10 μg·hr/mL within 96 hours of dosing.

The multiparticulates may be mixed or blended with one or more pharmaceutically acceptable materials to form a suitable dosage form. Suitable dosage forms include tablets, capsules, sachets, oral powders for constitution and the like.

The multiparticulates may also be dosed with alkalizing agents to reduce the incidence of side effects. The term “alkalizing agents”, as used herein, means one or more pharmaceutically acceptable excipients that will raise the pH in a constituted suspension or in a patient's stomach after being orally administered to said patient. Alkalizing agents include, for example, antacids as well as other pharmaceutically acceptable (1) organic and inorganic bases, (2) salts of strong organic and inorganic acids, (3) salts of weak organic and inorganic acids, and (4) buffers. Exemplary alkalizing agents include, but are not limited to, aluminum salts such as magnesium aluminum silicate; magnesium salts such as magnesium carbonate, magnesium trisilicate, magnesium aluminum silicate, magnesium stearate; calcium salts such as calcium carbonate; bicarbonates such as calcium bicarbonate and sodium bicarbonate; phosphates such as monobasic calcium phosphate, dibasic calcium phosphate, dibasic sodium phosphate, tribasic sodium phosphate (TSP), dibasic potassium phosphate, tribasic potassium phosphate; metal hydroxides such as aluminum hydroxide, sodium hydroxide and magnesium hydroxide; metal oxides such as magnesium oxide; N-methyl glucamine; arginine and salts thereof; amines such as monoethanolamine, diethanolamine, triethanolamine, and tris(hydroxymethyl)aminomethane (TRIS); and combinations thereof. Preferably, the alkalizing agent is TRIS, magnesium hydroxide, magnesium oxide, dibasic sodium phosphate, TSP, dibasic potassium phosphate, tribasic potassium phosphate or a combination thereof. More preferably, the alkalizing agent is a combination of TSP and magnesium hydroxide. Alkalizing agents are disclosed more fully for azithromycin-containing multiparticulates in commonly assigned U.S. patent application Ser. No. ______ (“Azithromycin Dosage Forms With Reduced Side Effects,” Attorney Docket No. PC25240), filed concurrently herewith.

The multiparticulates made by the inventive process may be post-treated to improve drug crystallinity and/or the stability of the multiparticulate. In one embodiment, the multiparticulates comprise azithromycin and a carrier, the carrier having a melting point of Tₘ° C.; the multiparticulates are treated after formation by at least one of (i) heating the multiparticulates to a temperature of at least about 35° C. and less than about (Tₘ° C.-10° C.), and (ii) exposing the multiparticulates to a mobility-enhancing agent. This post-treatment step results in an increase in drug crystallinity in the multiparticulates and typically an improvement in at least one of the chemical stability, physical stability, and dissolution stability of the multiparticulates. Post-treatment processes are disclosed more fully in commonly assigned U.S. patent application Ser. No. ______, (“Multiparticulate Compositions with Improved Stability,” Attorney Docket No. PC11900) filed concurrently herewith.

Without further elaboration, it is believed that one of ordinary skill in the art can, using the foregoing description, utilize the present invention to its fullest extent. Therefore, the following specific embodiments are to be construed as merely illustrative and not restrictive of the scope of the invention. Those of ordinary skill in the art will understand that known variations of the conditions and processes of the following examples can be used.

### EXAMPLE 1

Multiparticulates were made by a spray-drying process using the following procedure. First, 50 g of the HF grade of the carrier hydroxypropyl methyl cellulose acetate succinate having a concentration of acid and ester substituents of 3.2 meq/g carrier (HPMCAS-HF from Shin Etsu) and 4 g of NH₄OH were added to 455 g of distilled water to form a solution with a pH of greater than 8. To this solution was added 50 g of azithromycin dihydrate crystals having a degree of crystallinity of >99% to form a suspension of the azithromycin dihydrate in a solution of the HPMCAS-HF and high pH water. This suspension was stirred for 1 hour. The resulting suspension consisted of 8.94 wt % HPMCAS-HF, 8.94 wt % azithromycin dihydrate, 0.72 wt % NH₄OH, and 81.40 wt % water. The make-up of this suspension is summarized in Table 1 and it was spray-dried using the conditions given in Table 2 by continuously stirring the suspension to prevent settling of the suspended azithromycin dihydrate crystals and feeding it directly to a Niro 2-fluid atomizing nozzle with a 1-mm air gap using a peristaltic pump at a nominal rate of 40 g/min. Nitrogen at a flow rate of 193 g/min and a pressure of 40 psig was used to atomize the solution into a Niro PSD-1 spray-drying chamber. Drying nitrogen at 200° C. was introduced to the chamber at a rate of 1700 g/min. Drying gas and evaporated water exited the dryer at a temperature of 62° C. The resulting azithromycin-containing multiparticulates were collected using a cyclone. Analysis showed the multiparticulates to have a mean particle diameter of 26 μm. The multiparticulates comprised about 50 wt % azithromycin dihydrate and 50 wt % HPMCAS-HF. The concentration of acid and ester substituents on the carrier was calculated to be 3.2 meq/g azithromycin.

### EXAMPLE 2

Spray-dried multiparticulates having a mean particulate diameter of 35 μm were formed as in Example 1 with the exceptions noted in Tables 1 and 2. The multiparticulates of Example 2 comprised about 36.7 wt % azithromycin dihydrate and 63.3 wt % HPMCAS-HF. The concentration of acid and ester substituents on the carrier was calculated to be 5.5 meq/g azithromycin.

TABLE 1

| | Azithromycin | | | | | | |
|---|---|---|---|---|---|---|---|
| | Dihydrate | Carrier | | Solvent | | Additive | |
| Ex. | (g) | Type | (g) | Type | (g) | Type | (g) |
| 1 | 50 | HPMCAS- | 50 | Water | 455 | NH₄OH | 4 |
| | | HF | | | | | |
| 2 | 40 | HPMCAS- | 69 | Water | 580 | NH₄OH | 16 |
| | | HF | | | | | |

TABLE 2

| | | | | | Drying | Drying | Drying |
|---|---|---|---|---|---|---|---|
| | | Feed | Atomizing | | Gas | Gas | Gas |
| | | Suspension | Gas Flow | Atomization | Flow | Inlet | Outlet |
| | | Flow Rate | Rate | Pressure | Rate | Temp. | Temp. |
| Ex. | Carrier | (g/min) | (g/min) | (psig) | (g/min) | (° C.) | (° C.) |
| 1 | HPMCAS-HF | 40 | 193 | 40 | 1700 | 200 | 62 |
| 2 | HPMCAS-HF | 83 | 103 | 17 | 1860 | 250 | 72 |

The azithromycin release rate from the multiparticulates of Examples 1 and 2 was determined using the following procedure. A 750 mg sample of the multiparticulates was placed into a USP Type 2 dissoette flask equipped with Teflon-coated paddles rotating at 50 rpm. For Example 1 the flask contained 750 mL of 0.1 N HCl (pH 2) simulated gastric buffer held at 37.0±0.5° C. For Example 2, the flask contained 750 mL of 0.01 N HCl (pH 2) simulated gastric buffer held at 37.0±0.5° C. The multiparticulates were pre-wet with 10 mL of the simulated gastric buffer before being added to the flask. A 3 mL sample of the fluid in the flask was then collected at the elapse of 5,10, 15, 30, 45, 60, and 120 minutes following addition of the multiparticulates to the flask for Example 1; and at 5,15, 30, and 60 minutes for Example 2. The samples were filtered using a 0.45 μm syringe filter prior to analysis by HPLC (Hewlett Packard 1100, Waters Symmetry C8 column, 45:30:25 acetonitrile:methanol:25mM KH₂PO₄ buffer at 1.0 mL/min, absorbance measured at 210 nm with a diode array spectrophotometer).

TABLE 3

| | | Azithromycin |
|---|---|---|
| | Time | Released |
| Example | (min) | (%) |
| 1 | 0 | 0 |
| | 5 | 62 |
| | 10 | 74 |
| | 15 | 78 |
| | 30 | 83 |
| | 45 | 84 |
| | 60 | 84 |
| | 120 | 85 |
| 2 | 0 | 0 |
| | 5 | 40 |
| | 15 | 58 |
| | 30 | 60 |
| | 60 | 63 |

The multiparticles of Example 2 were then analyzed for the presence of azithromycin esters by LCMS. Samples were prepared by extraction with isopropyl alcohol at a concentration of 1.25 mg azithromycin/mL and sonicated for 15 minutes. The sample solutions were then filtered with a 0.45 μm nylon syringe filter. The sample solutions were then analyzed by HPLC using a Hypersil BDS C18 4.6 mm×250 mm (5 μm) HPLC column on a Hewlett Packard HP1100 liquid chromatograph. The mobile phase employed for sample elution was a gradient of isopropyl alcohol and 25 mM ammonium acetate buffer (pH approximately 7) as follows: initial conditions of 50/50 (v/v) isopropyl alcohol/ammonium acetated; the isopropyl alcohol percentage was then increased to 100% over 3 minutes and held at 100% for an additional 15 minutes. The flow rate was 0.80 mL/min. A 75 mL injection volume and a 43° C. column temperature were used.

A Finnigan LCQ Classic mass spectrometer was used for detection. The APCI source was used in positive ion mode with a selective ion-monitoring method. Calculations for the presence of azithromycin esters were made from the MS peak areas based on an external azithromycin standard and revealed the complete absence of azithromycin esters.

### EXAMPLE 3

Multiparticulates were made by a spray-coating process using the following procedure. First, 30 g of the low reactivity carrier hydroxypropyl cellulose containing virtually no acid or ester substituents (KLUCEL EF from Aqualon, Inc. of Wilmington, Del.) were dissolved into 800 g of distilled water. To this solution was then added 119.6 g of crystalline azithromycin dihydrate having a degree of crystallinity of >99%. The pH of the resulting coating solution was 9, indicating that the amount of azithromycin dihydrate dissolved in the solution was less than 1 mg/mL.

This coating solution was then sprayed onto 500 g of microcrystalline cellulose seed cores in a Glaft GPGC-1 fluidized-bed coating apparatus equipped with a Wurster column. The seed cores had a nominal diameter of 170 μm. The coating was applied by fluidizing the seed cores with 38 to 42 ft³l/min fluidizing nitrogen, heated to an inlet temperature of 52° C. to 55° C. The coating solution was sprayed onto the cores at a rate of 8 to 12 g/min using a two-fluid nozzle and an atomization air pressure of 2 bar. After 90 minutes of coating, the coating amounted to 19.2 wt % of the initial core weight. The cores therefore contained 12.8 mgA azithromycin per gram of coated cores.

The rate of release of azithromycin from these spray-coated multiparticulates was determined using the following procedure. A 1000 mg sample of the multiparticulates was placed into a USP Type 2 dissoette flask equipped with Teflon-coated paddles rotating at 50 rpm. The flask contained 750 ml of pH 6.8 phosphate buffer. The multiparticulates were pre-wetted with 10 mL of the phosphate buffer before being added to the flask. A 3 mL sample of the fluid in the flask was then collected at the elapse of 5, 10,15, 30, 60, and 120 minutes following addition of the multiparticulates to the flask. The samples were filtered using a 0.45-m syringe filter prior to analyzing via HPLC (Hewlett Packard 1100, Waters Symmetry C₈ column, 45:30:25 acetonitrile:methanol:25 mM KH₂PO₄ buffer at 1.0 ml/min, absorbance measured at 210 nm with a diode array spectrophotometer).

TABLE 4

| | Azithromycin |
|---|---|
| Time | Released |
| (min) | (%) |
| 0 | 0 |
| 5 | 92 |
| 10 | 94 |
| 15 | 96 |
| 30 | 98 |
| 60 | 99 |
| 120 | 100 |

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A liquid-based process for the formation of multiparticulates comprising the steps:
(a) forming a mixture comprising azithromycin, a pharmaceutically acceptable carrier, and at least one liquid having a boiling point of less than about 150° C.;
(b) forming particles from said mixture of step (a) by a method selected from
(i) atomization of said mixture, and
(ii) coating seed cores with said mixture; and
(c) removing a substantial portion of said liquid from said particles of step (b) to form said multiparticulates
wherein the following expression is satisfied:
[A]≦0.04/(1−x)
where [A] is the concentration of acid/ester substitution on the carrier in meq/g azithromycin, and x is the weight fraction of the azithromycin in said multiparticulates that is crystalline.

2. The process of claim 2 wherein the following expression is satisfied:
[A]≦0.02/(1−x).

3. The process of claim 2 wherein the following expression is satisfied:
[A]≦0.008/(1−x).

4. The process of claim 3 wherein the following expression is satisfied:
[A]≦0.004/(1−x).

5. The process of claim 1 wherein said liquid has a concentration of acid and ester substituents of less than 0.1 meq/g and is selected from the group consisting of water, an alcohol, an ether, a ketone, a hydrocarbon, a chlorocarbon, tetrahydrofuran, dimethylsulfoxide, N-methylpyrrolidinone, N,N-dimethylacetamide, acetonitrile and mixtures thereof.

6. The process of claim 5 wherein said alcohol is selected from the group consisting of methanol, ethanol, propanol and isomers thereof, and butanol and isomers thereof; said ketone is selected from the group consisting of acetone, methylethyl ketone and methylisobutyl ketone; said ether is selected from the group consisting of methyl tert-butyl ether, ethyl ether and ethylene glycol monoethyl ether; said chlorocarbon is selected from the group consisting of chloroform, methylene dichloride and ethylene dichloride; and said hydrocarbon is selected from the group consisting of pentane, hexane, heptane, cyclohexane, methylcyclohexane, octane and mineral oil.

7. The process of claim 5 wherein said liquid is water and includes a base selected from the group consisting of a hydroxide, a carbonate, a bicarbonate, a borate, an amine, a protein, an amino acid and mixtures thereof.

8. The process of claim 1 wherein steps (b) and (c) occur substantially simultaneously.

9. The process of claim 1 wherein said azithromycin is substantially in the form of the crystalline dihydrate.

10. The process of claim 9 wherein said azithromycin has a solubility in said liquid of less than about 10 mg/mL.

11. The process of claim 9 wherein water is added during at least one of steps (a), (b) and (c).

12. The process of claim 9 wherein step (c) is performed in a dryer selected from the group consisting of a tray dryer, a microwave dryer, a fluid bed dryer, a rotary dryer and a spray dryer.

13. The process of claim 12 including maintaining a level of humidity during step (c) which is greater than or equal to the activity of water of azithromycin in its crystalline state.

14. The process of claim 1 wherein the concentration of azithromycin esters in said multiparticulates is less than about 1 wt %.

15. The process of claim 14 wherein the concentration of azithromycin esters in said multiparticulates is less than about 0.5 wt %.

16. The process of claim 1 wherein steps (b) and (c) are conducted by spray-drying.

17. The process of claim 1 wherein step (b) is conducted by coating seed cores with said mixture to form coated seed cores, and step (c) is conducted by drying said coated seed cores.

18. The process of claim 1 wherein said multiparticulates comprise from about 20 to about 75 wt % of said azithromycin, from about 25 to about 80% of said carrier, and from about 0.1 to about 30 wt % of a dissolution enhancer.

19. The process of claim 18 wherein said multiparticulates comprise from about 35 to about 55 wt % of said azithromycin, from about 40 to about 65% of said carrier, and from about 0.1 to about 15 wt % of said dissolution enhancer.

20. The process of claim 19 wherein said multiparticulates comprise from about 45 to about 55 wt % of said azithromycin, and from about 45 to about 55% of said carrier.

21. The process of claim 18 wherein said carrier is selected from the group consisting of a wax, a glyceride, and mixtures thereof.

22. The process of claim 18 wherein said carrier is selected from the group consisting of synthetic wax, microcrystalline wax, paraffin wax, Carnauba wax, beeswax, glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate, and mixtures thereof.

23. The process of claim 18 wherein said dissolution enhancer is selected from the group consisting of surfactants, alcohols, sugars, salts, amino acids, and mixtures thereof.

24. The process of claim 18 wherein said dissolution enhancer is selected from the group consisting of poloxamers, polyoxyethylene alkyl ethers, polyethylene glycol, polysorbates, polyoxyethylene alkyl esters, sodium lauryl sulfate, sorbitan monoesters, stearyl alcohol, cetyl alcohol, polyethylene glycol, glucose, sucrose, xylitol, sorbitol, maltitol, sodium chloride, potassium chloride, lithium chloride, calcium chloride, magnesium chloride, sodium sulfate, potassium sulfate, sodium carbonate, magnesium sulfate, potassium phosphate, alanine, glycine, and mixtures thereof.

25. The process of claim 24 wherein said dissolution enhancer is a poloxamer.

26. The process of claim 25 wherein said carrier is a mixture of glyceryl mono-, di- or tribehenates.

27. The process of claim 26 wherein said azithromycin is substantially in the form of the crystalline dihydrate.

28. The product of the process of claim 1.
